(19) United States
(12) Patent Application Publication
(10) Pub. No.: US 2022/0188754 A1
(43) Pub. Date: Jun. 16, 2022

(54) DETECTION AND ALERTING SYTEM FOR PRECURSORS TO SPOILAGE OR SPONTANEOUS COMBUSTION AND THE METHOD THEREOF

(71) Applicant: Sipco Pty Ltd, Adelaide (AU)

(72) Inventor: Richard KELLY, Adelaide (AU)

(73) Assignee: Sipco Pty Ltd, Adelaide (AU)

(21) Appl. No.: 17/451,353

(22) Filed: Oct. 19, 2021

(30) Foreign Application Priority Data
Dec. 16, 2020 (AU) 2020904680

(51) Int. Cl. G06Q 10/08; G01K 13/10; G06Q 50/02

(52) U.S. Cl. CPC G06Q 10/087; G06Q 50/02; G01K 13/10

(57) ABSTRACT

A detection and alerting system for precursors to spoilage in haystacks caused by aerobic microbe activity. The system includes a plurality of temperature probes, measuring the temperature of the haystacks, at least one local communication system, communicating with the temperature probes about the temperatures of the haystacks. The system further includes an internet connected base communication module communicating with each of the local communication module, providing the temperature information of the haystacks; and alerting if the temperature information includes a first temperature indication such as a rapid increase in temperature.

### FIELD OF THE INVENTION

The present invention relates to system and method of detection and alert for precursors to spoilage and/or spontaneous combustion in haystacks due to aerobic respiration, deterioration and decomposition.

### BACKGROUND TO THE INVENTION

The process of spoilage in silage is an anaerobic process without Oxygen, which is normally called the fermentation process. For the fermentation process to occur, the moisture content has to be much higher than in the hay making process.

However, the process of spoilage in hay due to the aerobic processes which needs Oxygen is well known by those skilled in the art. The initial stage of this process is commonly called “curing”. For this aerobic process, the moisture content has to be much lower than is required for making silage.

One of the determining factors in hay making is the quantity and type of microbial content in the hay (bacteria, yeast and moulds—commonly called “bugs” in the hay industry) when it is stored. So long as there is moisture, water soluble carbohydrates (mainly sugars) and oxygen the microbes will grow in numbers, typically exponentially. Tests have shown that approximately 10 days after a mown crop has been cut down, the microbial count (usually measured as the number of colony forming units per gram “cfu per gram”) will have grown to a dangerous level, and the hay will have to be very dry (typically less than 14%) to be baled safely at these microbial count levels. If the hay is left down for a longer time, then this critical moisture level can be even lower and explains why some hay at much lower moisture contents (as low as 11%) has been known to spontaneously combust after it was left down for over 6 weeks before baling.

The microbes use the sugars for food and generate heat, carbon dioxide and moisture; hence the “increase” in moisture content measured several days after baling and referenced to the bales as “sweating”.

Typically, the hay is cut and windrowed and left for several days to dry. The hay dries rapidly in the first 24 to 36 hours. What appears to happen is the top and sides of the windrow dry much more than the centre and the bottom of the windrow (depending on weather and ground conditions). If this hay is baled, without being tedded out (a hay spreading process), or without conditioned windrows being flipped over after a few days, then the moisture tests from windrow samples will often show very variable moisture contents from samples just a short distance apart, and it is difficult to get an accurate average moisture content. Hay that is left in valleys, lower sections of the field and the outside double rows of fields, often have much heavier windrows that retain more moisture than the windrows in the remainder of the field. These wetter windrows and wet spots may lead to a more rapid breeding of the microbes.

When the moisture of a sample is analysed in the laboratory, the result is the total moisture content of the sample. What these results do not tell you is the distribution of the moisture within the plant itself (often called stem and dew moisture). Moisture in the nodes of some plants is much less slowly evaporated off than the moisture in the stems, which in turn is slower than the leaves. This is the main reason for using a conditioning or super conditioning machine, which crushes the stems and nodes and breaks them open to speed up the evaporation in the plant nodes and stems.

Failed grain cereal crops which have been cut down for hay have been a large source of spontaneous combustion events, mainly due to moisture contained in the nodes and immature grain heads curled up in the boot or swollen section near the top of the plant stem.

Thus even if the stem is dry, moisture and sugars in the nodes and immature heads can continue to encourage the build-up in the number of microbes. When the moisture in the nodes reduces to a suitable level the hay is usually termed “cured”. If you are using a hay preservative (for example an Oxygen scavenging Sulphur based product), the hay does not have to be cured to quite the same moisture content as with hay that has not had preservative applied.

The bulk density of the hay bales also plays a part in the propensity to spoil. Large rectangular hay bales which are typically pressed to a much higher bulk density than small rectangular hay bales or round bales are much more susceptible to aerobic spoilage.

The timing for cutting the hay is the same irrespective of using a hay preservative or not. Typically for legumes this is when there is 5 to 10 percent flower. For cereal it is at the late clear liquid stage or milk stage depending on the temperature to maximise grain formation, but not to the extent of getting grain drop during harvest of the hay.

Typically, the hay making process is as follows. After cutting, conditioning, and windrowing, the hay is tedded out for 24 to 36 hours (depending on the weather) to evenly apply the initial rapid drying process to the hay, then windrow back up again. The tedding is an extra procedure, but typically the value benefit in doing this is a higher metabolizable energy (ME), lower neutral detergent fibre (NDF), better leaf retention on the stem in the bale (i.e. better Relative Feed Value), better colour and better looking and smelling hay. Also bleaching won't be an issue in such a short time frame. Ideally hay should be baled within 5 to 7 days from cutting before microbial growth grows to dangerous levels.

Although many countries use the metric measurement system, the size of hay bales is typically still referred to by their imperial measurements in feet and inches.

Typically, the storage of large rectangular hay bales which has had a preservative applied is as follows. The hay is stacked in single rows in the shed no higher than 5 bales high for 4′×3′ and 4 bales high for 4′×4′ and about half a meter apart. After the stacks of bales have ceased changing temperature (typically at least 3 to 4 weeks), they may be stacked closer together. In some situations, hay baled with little or no dew moisture and elevated stem moisture (on dry down) should typically be left out in the field for approximately one week then follow the above stacking procedure.

Large hay producers typically have many haystacks in hay sheds. These hay sheds are typically separated by some distance, often measured in Kilometres to minimize the chances of a haystack fire destroying all of the hay produced.

Measuring the temperature of hay bales in haystacks has been carried out by hay farmers for decades. Typically, this is done with a pointed temperature probe. Such temperature probes have been on the market since the mid twentieth century.

Wireless connected temperature probes have also been on the market place for many decades. However, the cost of having individual telemetry links from each hay shed to an Internet based server is relatively high.

The object of this invention is to provide system and method thereof to address the above shortcomings or at least to provide a useful alternative.

### SUMMARY OF THE INVENTION

In a first aspect the invention comprises a detection and alerting system for precursors to spoilage in haystacks caused by aerobic microbe activity, comprises a plurality of temperature probes, measuring the temperature of the haystacks; at least one local communication system, communicating with the temperature probes about the temperatures of the haystacks; an internet connected base communication module, wherein communicating with each of the local communication module; providing the temperature information of the haystacks; and alerting if the temperature information includes a first temperature indication.

In preference the first temperature indication comprises the temperature of the hay bale of the haystack; and/or the rate of temperature increase per unit time.

In preference the local communication system comprises a temperature receiver, receiving the temperatures of the haystacks from the temperature probes via wireless or wired connections; and a local communication module, communicating with the temperature receiver via wireless or wired connections.

In preference the base communication module comprises a collection sub-module, collecting the temperature information of each embedded temperature probe from the relevant local communication module and/or the temperature information of each surface temperature probe from the relevant base communication module; an alerting sub-module, determining whether the temperature of the hay bale of the haystack exceeds a first set point and/or the rate of temperature rise per unit time of the hay bale exceeds a second set point; and a transmission sub-module, providing the temperature information of each haystack to Internet connected electronic devices via an Internet connected server by SMS or emails or notifications.

In preference the collection sub-module of the base communication module collects the information about weather conditions, comprising air temperature, humidity, wind speed, wind direction, solar radiation, and barometric pressure.

In preference the temperature probes have the configuration of wireless spikes, wired spikes, wireless flat sensors or wired flat sensors.

In preference the temperature probes are located between the bales of the haystacks or close to the top surface of the haystacks.

In preference the system further comprises a power supply module, providing power for the local communication system.

In preference the system further comprises an Internet connected applications for the Internet connected electronic devices, displaying the temperature information and/or the first temperature indication of the temperature information.

In a further form of the invention there is proposed a detection and alerting method for precursors to spoilage in haystacks, comprising the steps of detecting the temperature of the haystacks using a plurality of temperature probes; communicating with the temperature probes about the temperatures of the haystacks and providing haystack temperature information and alerting if the temperature information includes a first temperature indication.

In preference the method further comprises measuring the temperature of the hay bale of the haystack; and/or the rate of temperature increase per unit time of the hay bale.

It should be noted that any one of the aspects mentioned above may include any of the features of any of the other aspects mentioned above and may include any of the features of any of the embodiments described below as appropriate.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows.

FIG. 1 is a schematic view of the detection and alerting system according to an embodiment of the present invention.

FIG. 2 is a schematic close-up view of the base hay shed of the detection and alerting system according to an embodiment of the present invention.

### DETAILED DESCRIPTION OF THE INVENTION

The following detailed description of the invention refers to the accompanying drawings. Wherever possible, the same reference numbers will be used throughout the drawings and the following description to refer to the same and like parts. Dimensions of certain parts shown in the drawings may have been modified and/or exaggerated for the purposes of clarity or illustration.

Foremost, the basis of the present invention is that the cost of having individual telemetry links from each hay shed to an Internet based server can be reduced by having each hay shed wirelessly connected to a base hay shed and that base hay shed connect to an internet connected server.

It is not the intention of this detection and alerting system to convey to operators what hay temperatures are at dangerous levels. The temperatures at which the hay reaches various stages of decomposition due to the aerobic microbial action is highly likely to vary with, but not limited to, crop type, baling conditions, haystack construction and moisture content. What it is determined is that the rate of heating per unit time (typically per hour) is the best indicator, combined with the absolute temperature values.

In some embodiments of the present invention, the detection and alerting system 10 for precursors to spoilage or spontaneous combustion comprises a base communication module 32 located on a base hay shed 22 and at least one local communication modules 30 located on the base hay shed 22. If there are non-base hay sheds 50, then they will also have at least one local communication module 30 respectively.

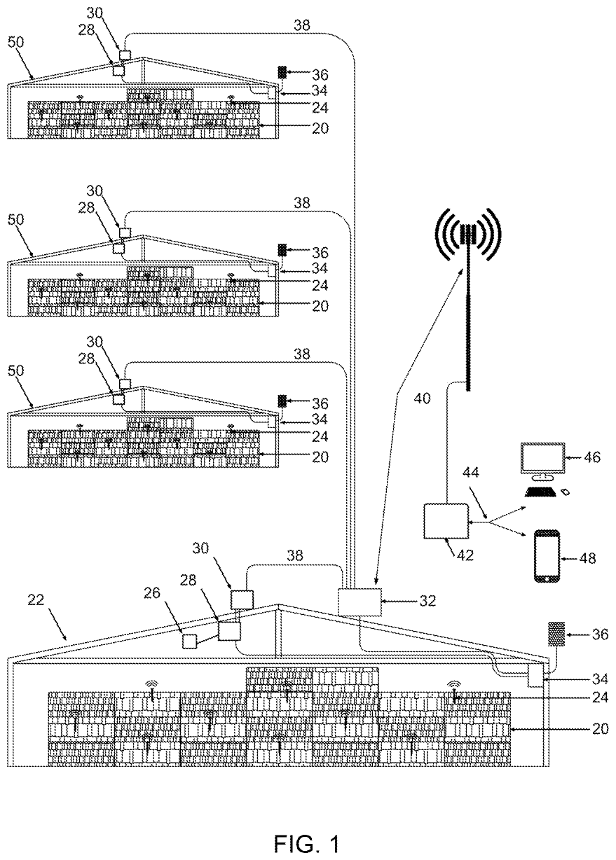

Referring to FIG. 1, in a preferred embodiment, there are four hay sheds. One of them on the bottom is the base hay shed 22 whereas the rest are the non-base hay sheds 50. Each hay shed has a local communication module 30.

The base communication module 32 typically has a cellular modem to communicate data to and from a cellular wireless communication link 40 and on to an internet server 42. Said base communication module 32 also communicates via a local wireless communication link 38, typically a wide area network (wan) wireless system known as LoRaWAN to each local communication module 30 on each hay shed, including the base hay shed 22 and three local non-base hay sheds 50.

A local communication system comprises a temperature receiver 28 which receives the temperatures of the haystacks 20 from temperature probes 24, 26 via wireless or wired connections and a local communication module 30 which communicates with the temperature receiver 28 via wireless or wired connections. The local communication module 30 receives its power from the power supply module 34 and communicates with the temperature receiver 28. The temperature receiver 28 also receives its power from the power supply module 34. The local communication module 30 and the temperature receiver 28 can also have their respective power supply unit, the specific form of which should be limited to the embodiment in FIG. 1.

Temperature probes 24, 26 are used to measure the temperature of the haystacks 20. Embedded temperature probes 24 which are used to measure the temperature of hay bales are embedded in the haystacks 20. Methods of stacking hay vary, but commonly use fork lifts with hydraulically operated arms to grab multiple hay bales at a time. Therefore, in some preferred embodiments, flat temperature probes laid down between bales may be the preferred sensor locating method.

One or more infrared sensors namely surface temperature probes 26 (sensor and probe are used interchangeably hereafter for convenience) that may be used to measure the temperature of the haystacks 20 can be installed in a place close to the top surface of the haystacks 20. The top surface temperature of the hay is used as a reference temperature when an operator sets temperature set points for triggering alerts and/or to enhance the temperature measurement profile.

Irrespective of using an insertion sensor probe or a sensor probe laid down between bales, it is preferred that each sensor be attached to a high visibility ribbon so that when the haystack is dismantled, the location of each temperature sensor is more easily located.

While the base communication module 32 collects the temperature information of each embedded temperature probe 24 from the local communication module 30, it determines if the temperature information includes a first temperature indication. The first temperature indication comprising the temperature of the hay bale of the haystack and/or the rate of temperature rise per unit time of the hay bale. If so, then the detection and alerting system 10 will alert.

Further, the base communication module comprising a collection sub-module, collecting the temperature information of each embedded temperature probe 24 from the relevant local communication module 30 and/or the temperature information of each surface temperature probe 26 from the base communication module 32. An alerting sub-module (not shown in the Figs), determining whether the temperature of the hay bale of the haystack exceeds a first set point and/or the rate of temperature rise per unit time of the hay bale exceeds a second set point. And a transmission sub-module, providing the temperature information of each haystack 20 to internet connected electronic devices via an internet connected server 42 by SMS or emails or notifications. Said first and second set point can be set by the operator based on the reference temperature detected by the surface temperature probes 26 or set by the server 42 automatically.

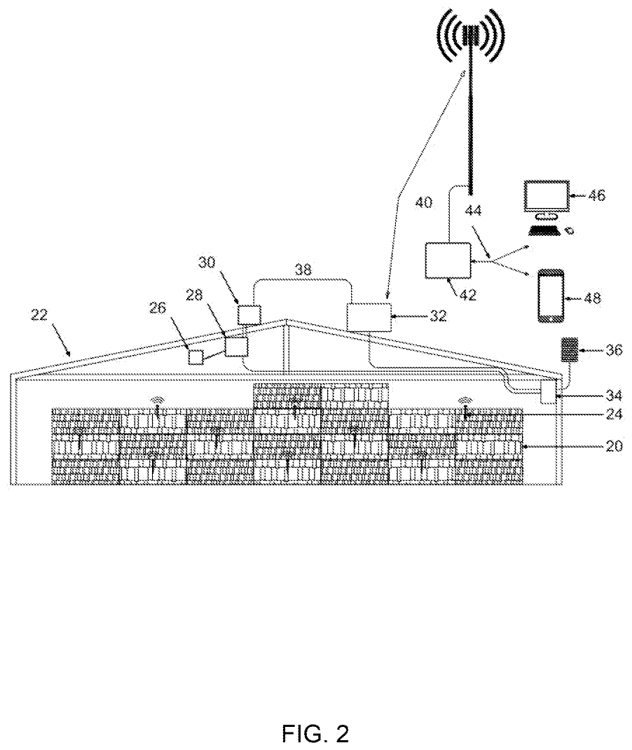

Referring to the preferred embodiment shown in FIGS. 1 and 2, there is only one surface temperature probes 26 beneath the roof of the base hay shed 22 while many embedded temperature probes 24 are distributed in the haystacks 20 both in the base hay shed 22 and the local non-base hay sheds 50. Four embedded temperature probes 24 on the right of the hay sheds 22, 50 are marked in FIG. 1. Each temperature probe 24, 26 communicates with the temperature receiver 28. Communication between the temperature probes and the temperature receiver 28 may be wired or wireless. Specifically, the temperature probes 24, 26 to measure the rise in temperature of the hay caused by aerobic microbial activity may be designed in any number of configurations. Such configurations may include wireless spiles, wired spikes, wireless flat sensors between bales or wired flat sensors between bales.

When the communication between the temperature probes 24, 26 and the temperature receiver 28 is wireless, then the operating frequency is required to be low enough to penetrate through damp hay without attenuating the signal to the extent that the temperature date cannot be read.

The wireless connected temperature probes 24, 26 typically include a battery power source. To maintain low power consumption and thus provide longevity of battery supply service the temperature probes 24, 26 operate on a low quiescent current until woken up at intervals typically measured in hours to read a temperature value and communicate said temperature value to the temperature receiver 28. After receiving the temperature value, the temperature receiver 28 sends it to the local communication module 30 on which shed the temperature receiver 28 is located. Then the local communication module can send the temperature value coming from the temperature receiver 28 to the local communication module 30 at intervals or in real time.

In order to locate the tested haystack 20, each of the temperature probes 24, 26 have their own unique identity. Typically, when each temperature probes 24, 26 is deployed in the haystack 20 its location is noted in an application located in an internet connected server 42. Said application may be accessed from an internet connected computer 46 or an internet connected smartphone 48 to display the hay temperatures in a graphical way in the location at which the temperatures were read. The connected computer 46 or the smartphone 48 is connected to the server 42 via wireless or wired internet connections 44.

Further, the base communication module 30 may further include sensors to measure environmental parameters and pass the values of the said parameters on through the local communication system and the base communication module 32 to be stored in the internet connected server 42. Said parameters may include, but not be limited to air temperature, humidity, wind speed, wind direction, solar radiation and barometric pressure. The base communication module 32 can also determine if the alerts of the detection and alerting system 10 should be triggered using these parameters individually or together with the temperature information of the haystack 20 discussed above.

Temperature set points or set points for other environmental parameters may be configured by the operator to provide alerts when hay temperatures rise above said set points. Said alerts may take the form of, but not limited to, Small Messaging Service (SMS), emails and smartphone Notifications.

In some other embodiments, the detection and alert system 10 as described above, may be reduced to include only one base hay shed 22 and one haystack 20 without compromising the intended application of the system.

The reader will now appreciate the present invention which provides system and method of the detection and alerting system 10.

### LIST OF COMPONENTS

The drawings include the following integers.
10 a detection and alerting system
20 a haystack
22 a base hay shed
24 an embedded temperature probe
26 a surface (infrared) temperature probe
28 a temperature receiver
30 a local communications module
32 a base communications module
34 a power supply module
36 photovoltaic cells
38 a local wireless communications link
40 a cellular wireless communications link
42 an Internet connected server
44 internet connections
46 an Internet connected computer
48 an Internet connected smartphone
50 local non-base hay sheds

Further advantages and improvements may very well be made to the present invention without deviating from its scope. Although the invention has been shown and described in what is conceived to be the most practical and preferred embodiment, it is recognized that departures may be made therefrom within the scope of the invention, which is not to be limited to the details disclosed herein but is to be accorded the full scope of the claims so as to embrace any and all equivalent devices and apparatus. Any discussion of the prior art throughout the specification should in no way be considered as an admission that such prior art is widely known or forms part of the common general knowledge in this field.

In the present specification and claims (if any), the word “comprising” and its derivatives including “comprises” and “comprise” include each of the stated integers but does not exclude the inclusion of one or more further integers.

1. A detection and alerting system for precursors to spoilage in haystacks caused by aerobic microbe activity, said system comprising:
a plurality of temperature probes measuring a temperature of the haystacks;
at least one local communication system, communicating with the plurality of temperature probes about the temperature of the haystacks; and
an internet connected base communication module communicating with the at least one local communication system; providing temperature information of the haystacks; and alerting if the temperature information includes a first temperature indication.

2. The system of claim 1, the first temperature indication comprising:
the temperature of a hay bale of the haystacks; and/or
a rate of temperature increase per unit time.

3. The system of claim 1, the at least one local communication system comprising:
a temperature receiver, receiving the temperatures of the haystacks from the plurality of temperature probes via wireless or wired connections; and
a local communication module, communicating with the temperature receiver via wireless or wired connections.

4. The system of claim 1, the base communication module comprising:
a collection sub-module, collecting the temperature information of each embedded temperature probe of the plurality of temperature probes from a relevant local communication module and/or the temperature information of each surface temperature probe of the plurality of temperature probes from the relevant local communication module;
an alerting sub-module, determining whether the temperature of a hay bale of the haystacks exceeds a first set point and/or a rate of temperature rise per unit time of the hay bale exceeds a second set point; and
a transmission sub-module, providing the temperature information of each haystack of the haystacks to internet connected electronic devices via an internet connected server by SMS or emails or notifications.

5. The system of claim 4, wherein the collection sub-module of the base communication module collects information about weather conditions, comprising air temperature, humidity, wind speed, wind direction, solar radiation, and barometric pressure.

6. The system of claim 1, wherein the plurality of temperature probes has the configuration of wireless spikes, wired spikes, wireless flat sensors or wired flat sensors.

7. The system of claim 1, wherein the plurality of temperature probes is located between bales of the haystacks or close to a top surface of the haystacks.

8. The system of claim 1, further comprising:
a power supply module, providing power for the at least one local communication system.

9. The system of claim 1, further comprising:
internet connected applications for internet connected electronic devices, said internet connected applications displaying the temperature information and/or the first temperature indication of the temperature information.

10. A detection and alerting method for precursors to spoilage in haystacks, comprising:
detecting a temperature of the haystacks using a plurality of temperature probes;
communicating with the plurality of temperature probes about the temperature of the haystacks and providing haystack temperature information; and
alerting if the temperature information includes a first temperature indication.

11. The method of claim 10, the first temperature indication comprising:
measuring the temperature of a hay bale of the haystacks; and/or a rate of temperature increase per unit time for the hay bale.
